(19) United States
(12) Patent Application Publication
(10) Pub. No.: US 2026/0105779 A1
(43) Pub. Date: Apr. 16, 2026

(54) WELLBEING MONITORING

(71) Applicant: British Telecommunications Public Limited Company, London Greater London (GB)

(72) Inventors: Beum Seuk LEE, London (GB); David YEARLING, London (GB); Bryan SCOTNEY, London (GB)

(21) Appl. No.: 19/114,752

(22) PCT Filed: Aug. 22, 2023
(86) PCT No.: PCT/EP2023/072995

§ 371 Date: Mar. 24, 2025

(30) Foreign Application Priority Data
Sep. 28, 2022 (EP) 22198424.8
Sep. 28, 2022 (GB) 2214181.6

(51) Int. Cl. G06V 40/20; G06V 10/34; G06V 10/764; G06V 40/10; G10L 15/22; G16H 20/00

(52) U.S. Cl. CPC G06V 40/20; G06V 10/34; G06V 10/764; G06V 40/10; G10L 15/22; G16H 20/00; G10L 2015/227

(57) ABSTRACT

Sedentary behaviours of a person may be monitored for well-being. Captured sensor data depicting at least one sedentary person, and values of configuration parameters of a capture device used to capture the sensor data are received. The captured sensor data is processed to compute skeletal data of the person. The skeletal data is input to a classifier to obtain classification data, for a plurality of classes related to behaviour of the person. The configuration parameters are stored in association with the classification data and patterns are detected in the stored classification data. Values of the configuration parameters are adjusted using the detected patterns.

The present disclosure relates to monitoring sedentary behaviours for wellbeing.

### BACKGROUND

There is an increasing need for automated wellbeing monitoring of all age groups. Wellbeing monitoring involves obtaining sensor data about behaviour of a person and, with their consent, providing the sensor data or information derived from the sensor data to the person or their carer.

Wellbeing monitoring is found to be particularly effective for encouraging and promoting healthy behaviours such as exercise and sleep.

The examples described herein are not limited to examples which solve problems mentioned in this background section.

### SUMMARY

Examples of preferred aspects and embodiments of the invention are as set out in the accompanying independent and dependent claims.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A first aspect of the disclosed technology.

In some preferred example embodiments.

Another aspect of the disclosed technology comprises

It will also be apparent to anyone of ordinary skill in the art, that some of the preferred features indicated above as preferable in the context of one of the aspects of the disclosed technology indicated may replace one or more preferred features of other ones of the preferred aspects of the disclosed technology. Such apparent combinations are not explicitly listed above under each such possible additional aspect for the sake of conciseness.

Other examples will become apparent from the following detailed description, which, when taken in conjunction with the drawings, illustrate by way of example the principles of the disclosed technology.

### BRIEF DESCRIPTION OF THE DRAWINGS

FIG. 1 is a schematic diagram of a sedentary behaviour tool;

FIG. 2 shows more detail of a sedentary behaviour tool;

FIG. 3 is a flow diagram of a method performed by an activity monitor of a sedentary behaviour tool;

FIG. 4 is a flow diagram of a method performed by a sedentary behaviour tool;

FIG. 5 is a schematic diagram of a computing device implementing a sedentary behaviour tool.

The accompanying drawings illustrate various examples. The skilled person will appreciate that the illustrated element boundaries (e.g., boxes, groups of boxes, or other shapes) in the drawings represent one example of the boundaries. It may be that in some examples, one element may be designed as multiple elements or that multiple elements may be designed as one element. Common reference numerals are used throughout the figures, where appropriate, to indicate similar features.

### DETAILED DESCRIPTION

The following description is made for the purpose of illustrating the general principles of the present technology and is not meant to limit the inventive concepts claimed herein. As will be apparent to anyone of ordinary skill in the art, one or more or all of the particular features described herein in the context of one embodiment are also present in some other embodiment(s) and/or can be used in combination with other described features in various possible combinations and permutations in some other embodiment(s).

The inventors have recognized a need for monitoring sedentary behaviour of people, especially older adults, for wellbeing purposes.

The inventors have recognized that using internet of things (IOT) sensors such as passive infrared (PIR) sensors, contact sensors, pressure sensors instead of using visual sensors leads to making inferences based on rudimentary information, giving unreliable or unsophisticated analysis outcomes. The present technology leverages visual loT sensors such as video cameras, thermal cameras, laser imaging detecting and ranging (LADAR) sensors, for wellbeing monitoring to enrich the information and to increase the inference accuracy. In some cases the sensor data is used to carry out in-depth analysis such as gait analysis and/or posture trend changes over time.

Some approaches to wellbeing monitoring focus on activities instead of sedentary activities. The present technology monitors prolonged inactivity which can be a great indicator of a person's wellbeing and thus can affect mental and physical health.

Some approaches carry out activity monitoring through wearable devices such as pedometers, wrist worn sensors, and smartphone onboard accelerometers. Wearable devices for monitoring levels of activity rely heavily on accelerometer technology. A pre-determined threshold for the amount of movement over a set duration of time may be used to classify periods of sedentary behaviour, and machine learning approaches may be used to identify specific types of sedentary behaviour from accelerometer data. Whilst accelerometer-based approaches demonstrate feasibility and utility, enabling continuous monitoring indoors and outdoors, there are some limitations, and it is reported that one-third of users abandon wearable tracker devices after a few months. Although wearables are useful, there are many limitations in their uses, especially monitoring activities and gross/fine movements that are related to wellbeing.

The present technology enables monitoring of sedentary behaviour without the need for wearable sensors. Thus, there is no need for a user to remember to wear a sensor device and there is no discomfort to a user from a wearable sensor.

The present technology is suitable for use in a resource constrained computing device, such as an edge computing device, making it suitable for use in domestic environments.

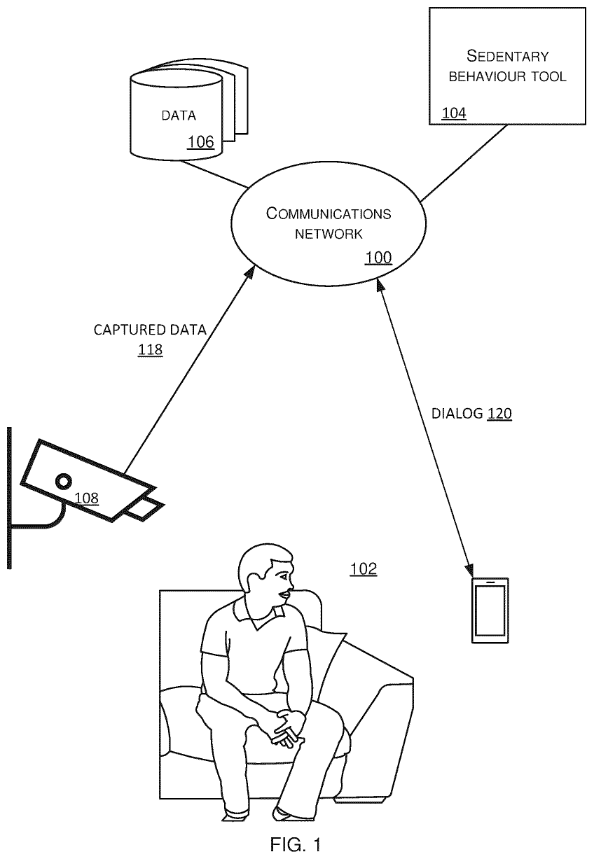

FIG. 1 is a schematic diagram of a sedentary behaviour tool 104 which is computer implemented. The sedentary behaviour tool is in communication with one or more capture devices 108 via a communications network 100 such as the internet, a wireless communications network, or any communications network. In some cases, the sedentary behaviour tool is a computing device in a home of the person and is a resource constrained computing device. In some cases, the sedentary behaviour tool is deployed in the cloud or at a computing device remote from the person 102.

The capture device 108 is any capture device for capturing sensor data depicting a person 102. A non-exhaustive list of examples of capture device is: video camera, red green blue camera, web camera, LADAR sensor, depth camera, time of flight camera. The capture device 108 is not worn by the person and in some cases is fixed to a wall of a room where the person is living. Although FIG. 1 shows one capture device 108 there may be a plurality of capture devices in practice. In various examples the capture device is an optical sensor.

The capture device 108 has one or more configurable parameters such as field of view, zoom, direction, exposure, focus or other capture device parameters. The configurable parameter values may be set by sending instructions to the capture device 108 from another entity such as the sedentary behaviour tool 104 or an intermediate computing device.

Captured data 118 from the capture device 108 is sent to the sedentary behaviour tool 104 via the communications network. The captured data 118 may be sent in encrypted form and/or compressed form.

The sedentary behaviour tool 104 receives the captured data 118 and uses it to monitor sedentary behaviour of the user and optionally to control an automated dialog 120 with the person 102 via a smart phone or other computing device in the vicinity of the user 102. The automated dialog is arranged to facilitate wellbeing of the person such as by triggering an alert in the event of an adverse health incident and/or encouraging healthy behaviours.

The sedentary behaviour tool 104 may have access to data 106 via communications network 100. The data comprises data about groups of users as explained in more detail below.

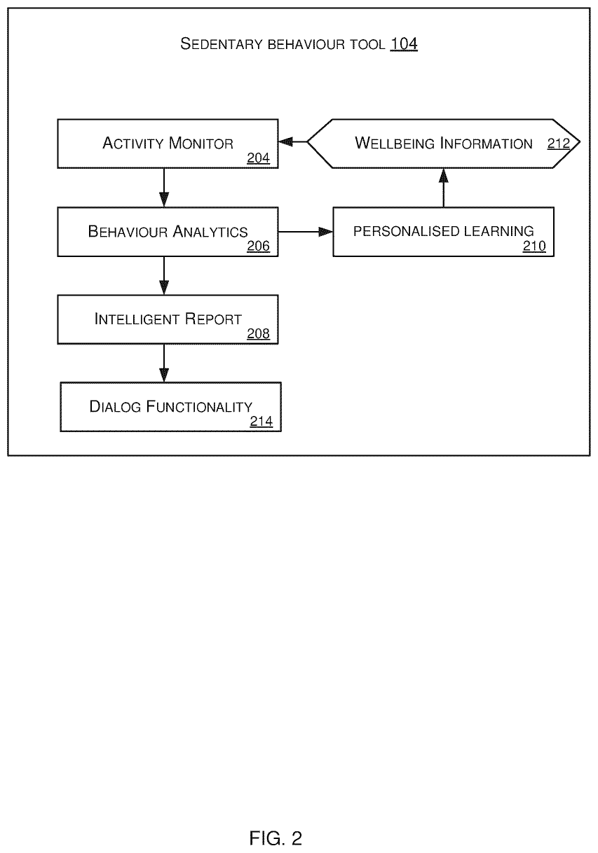

FIG. 2 shows more detail of a sedentary behaviour tool 104 such as that of FIG. 1. The sedentary behaviour tool 104 comprises an activity monitor 204, behaviour analytics 206, intelligent report functionality 208, dialog functionality 214 and personalised learning functionality 210. The sedentary behaviour tool 104 generates wellbeing information 212. The wellbeing information is provided as feedback to the activity monitor 204 as shown.

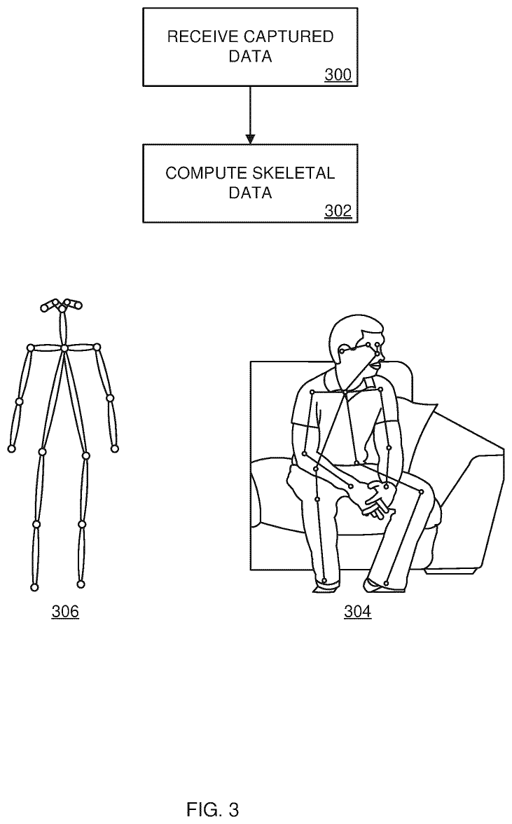

FIG. 3 is a flow diagram of a method performed by an activity monitor 204 of a sedentary behaviour tool 104 such as that of FIG. 1 or FIG. 2. The activity monitor 204 receives 300 captured data from one or more capture devices such as the capture device shown in FIG. 1. The captured data depicts a sedentary person such as the sedentary person illustrated in FIG. 1. The activity monitor 204 computes 302 skeletal data from the captured data. The skeletal data comprises the 2D locations in a frame of captured sensor data of a plurality of joints of a skeleton model 306. The 2D locations are computed by fitting the skeleton model 306 to the frame of captured sensor data such as by template matching or using a commercially available machine learning skeletal tracker. Depending on the particular captured data, some of the joints of the skeleton model may be occluded. Once the 2D locations of a plurality of joints of the skeleton model 306 are known, lengths of lines between the 2D locations of the joints are computed as well as angles between the lines and optionally ratios of the lines.

In some cases the skeletal data is used to detect more than one person and to store data about social interaction events when more than one person is present.

The inventors have found several benefits in using only the skeletal data instead of full image data from optical sensor devices. Although the skeletal data is compact, it is found to provide signatures for sedentary behaviour analytics, such as joint positions, angles between the lines connecting the joints, the lengths of the lines connecting the joints. There is increased security as the skeletal data collected from the optical sensor devices hide the information that could be sensitive otherwise. The skeletal data also brings computational economy. As the volume of the skeletal data is much smaller compared with full video data, a computing device to handle the data can be lightweight, small in size, low in its power consumption, computationally not expensive, and with less demanding requirements. For example, an edge computing device is sufficient to provide enough computing power for the present technology while placing compute and analytics close to where data is created.

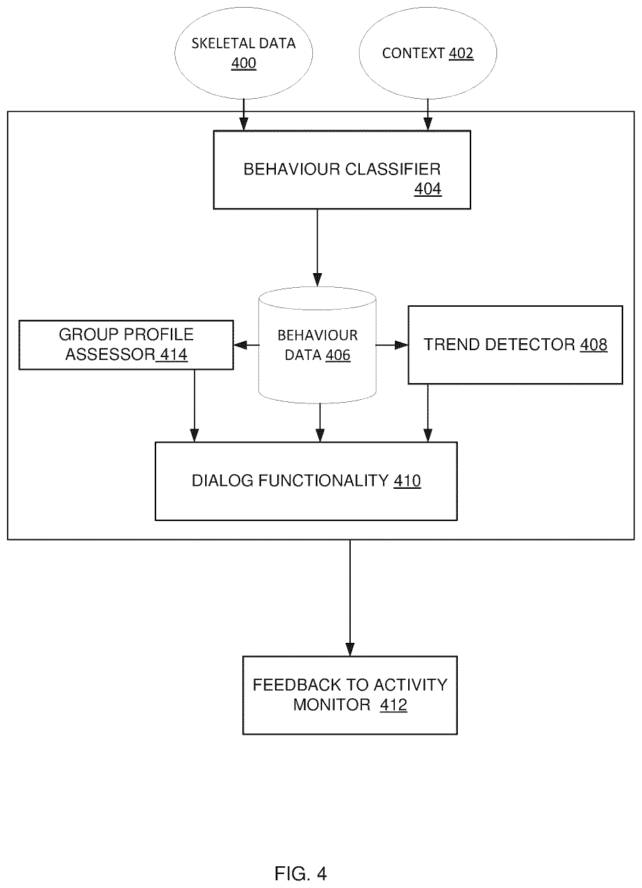

FIG. 4 is a flow diagram of a method performed by a sedentary behaviour tool 104 such as that of FIG. 1.

With reference to FIG. 4, skeletal data 400 computed by the activity monitor 204 is input to a behaviour classifier 404. Context 402 is optionally input the behaviour classifier. A non-exhaustive list of examples of context is one or more of: location of a room from which the captured sensor data was received, the time of the day, an identity of the person.

The behaviour classifier is any computing functionality for assigning a skeletal data instance amongst a plurality of classes of behaviour. The behaviour classifier is a trained machine learning model in some cases, such as a random decision forest, a neural network or a support vector machine. In some cases the behaviour classifier is a rule based system which uses rules configured by an operator in order to assign a skeletal data instance amongst a plurality of classes of behaviour, optionally taking into account context.

In some examples the classifier outputs a confidence value with each classification to indicate how likely the classification is to be correct.

In an example where the classifier is a neural network, the neural network is a multi-layer perceptron which has been trained using supervised learning. The training data comprises skeletal data labelled by human judges with behaviour class labels. The training is done using backpropagation.

A non-exhaustive list of example classes of behaviour is now given.

Behaviour class
Sleep
Napping
Dozing off
Awake but doing nothing specific
Knitting
Looking out the window
Sitting down
Lying down
Watching TV
Reading
Using a smartphone
Listening to the radio
Playing a video game
Interacting with a pet
On a call
Chatting with someone
Fallen down
Having a seizure
Having a heart attack
Suffering from chest pain
Having a headache
Smoking
Drinking alcohol
Having a snack
Having a meal
Having a cup of tea/coffee
Scratching oneself
Fidgeting or restless

The output from the behaviour classifier 404 is one or more classes of behaviour that the skeletal data 400 is assigned to, optionally taking into account the context 402. The output is stored in behaviour data store 406 such as a memory, database or other data store.

The behaviour classifier 404 operates repeatedly over time, as instances of skeletal data 400 arrive at the behaviour classifier from the activity monitor. In some cases the behaviour classifier 404 operates at or above a frame rate of the image capture device. Thus behaviour data accumulates in the behaviour data store 406.

A trend detector 408 has access to the data in the behaviour data store 406 and searches for one or more of: patterns in the behaviour data, anomalies in the behaviour data, trends in the behaviour data. Results from the trend detector 408 are available to dialog functionality 410.

By using the trend detector 408, it is possible to determine habitual postures and behaviours of a person and use those to identify or distinguish subjects.

By using the trend detector 408, it is possible to leverage historical data for trend analysis and wellbeing deterioration detection; not only the current data but also historical data can be used to detect significant changes in wellbeing status.

By using the trend detector 408 it is possible to achieve social interaction analysis. Interactions among subjects is analysed to measure social wellbeing status. For example, where a person is observed to spend a good portion of an afternoon sitting down (which can be interpreted as a negative sedentary behaviour) if the person is interacting with someone else at that time, there is a positive behaviour in the interest of the subject's social need.

By using the trend detector 408 it is possible to detect posture changes as an indicator of pathological issues. In an example, detect pathological issues by monitoring gradual changes in posture. For example, the subject may start to sit leaning toward his or her right because his or her old back problem has resurfaced.

A group profile assessor 414 has access to the data in the behaviour data store 406. The group profile assessor 414 knows about group profiles which are information about clusters of similar users of the sedentary behaviour tool. The group profile assessor 414 searches for one of the group profiles which is a closest match to the behaviour data. The group profile assessor then sends data about the closest matching group profile to the dialog functionality 410.

Dialog functionality 410 is any commercially available chat bot which is adapted to use information from the group profile assessor 414, trend detector 408 and behaviour data store 406.

The dialog functionality 410 utilises its inputs to converse with users in a friendly and intelligent way. The dialog functionality 410 is able to send alerts, to send highlights and to send predictions as now explained.

Alerts are made by the dialog functionality 410 where significant events that require prompt interventions such as a fall, a seizure, an intruder invasion, sleepwalking are detected. Alerts are sounds or messages made or triggered by the dialog functionality.

Highlights are made by the dialog functionality 410 to give indications of wellbeing status changes. For example, the subject has shown a pattern of increasing nap times during the day, possibly indicating not having sufficient sleeps at night.

Predictions are made by the dialog functionality 410 to indicate wellbeing status forecasting based on various aspects of the sedentary behaviours in the historical data.

As mentioned above, the dialog functionality receives input from the group profile assessor 414 such as information about a profile of a group that the person is most similar to. That is, the tool can profile each user by comparing him or her with other users and interacts with the user in the most effective way that has been proven to be successful among the users in his or her specific profile group. The profile data of the group the person is most similar to may comprise data about an effective way to dialog with users 20 in the group.

The dialog functionality 410 initiates a conversation with the user. The main goal of the dialog is to provide the user with informative suggestions so as to improve their wellbeing by using different types of interventions. The tool monitors behaviour changes after the dialog to discover which dialogs are effective for specific users and adapts the style, the frequency, the verbosity of the dialog, and the types and the intensities of the interventions accordingly.

Information from one or more of the group profile assessor 414, trend detector 408, dialog functionality 410 is used to create and send feedback 412 to the activity monitor. In an example, the feedback comprises values of configuration parameters of capture devices which are associated with a successful behaviour classification for a particular user or group of users. In another example, the feedback comprises values of parameters of a skeleton model which are associated with successful behaviour classification for a particular user or group of users.

The feedback 412 is used by the activity monitor to adjust configuration parameters of the capture device(s) and/or to adjust the skeleton model 306. In an example, a field of view of the capture device is adjusted to be close to a value associated with captured sensor data which yielded accurate behaviour data in the past. In another example, a number of joints in the skeleton model 306 is reduced (or increased) to match a number of joints which yielded accurate behaviour data in the past. When the person is sedentary in a particular position one or more joints may be occluded so that it is possible to reduce the number of joints in the skeleton model. The accuracy of the behaviour data is assessed by checking whether the behaviour classifier was able to classify skeletal data or whether no classification was possible, or by using confidence data associated with the classifications.

The tool improves itself in understanding the subjects through adaptive improvements in activity monitoring according to individual wellbeing status and the feedback from the dialog functionality 410. The tool is able to adjusting the number of skeleton points for more or less detailed information. The tool is able to change the angles or zoom levels of the optical sensor devices to capture the most occupied or active areas of the location. The tool is able to re-evaluate existing behaviours and learn new behaviours that are repeated enough to form patterns. The tool is able to use the analysis findings from other subjects to understand and to predict the wellbeing.

Lastly, coupled with the available information about the user (the age, the height, the weight, the medical records, the family health history, etc.), the wellbeing-related information about each user such as the daily activities including sedentary activities, the long term changes in activities, risky events (e.g. falls, seizure, sleep walking, etc.), and the history of the compliance/non-compliance with the recommendations from the system is used to analyse the wellbeing of the user and to profile the user accordingly. The profile information from the analysis result is recorded and updated for the carer(s) to take necessary interventions accordingly.

Example dialogs made by the dialog functionality 410 are now given.

```
  Emergency Situation where the user fell down and got injured
<System detects ‘Fallen down’ behaviour with the confidence of 97%>
System: Hello Mr. Johnson, are you alright?
      It seems like you are fallen down.
<No response detected from the user>
Should I contact your son, Dave for help?
Mr. Johnson: Yes, please.
System: Sure. I just sent Dave a message regarding your fall.
    Is there anything else I can do for you?
Mr. Johnson: No, thank you. That's it for now.
System: You are welcome. I will keep an eye on you, just in case.
      Please, let me know if you need anything else.
<System records the ‘Fallen down’ event>
Emergency Situation where the user is having a seizure and not responding
<System detects ‘Having a seizure’ behaviour with the confidence of 91%>
System: Hello Mr. Johnson, are you Okay?
It seems like you are having a seizure.
<No response detected from the user>
System: Should I contact an emergency service now?
<Still no response detected from the user>
<System detects ‘Having a seizure’ behaviour with the confidence of 96%>
System: I just sent a message for an emergency help.
I also informed Dave about your seizure by a text message as you have requested
previously.
<System records the ‘Seizure’ event>
Intervention
<System notices the user is spending too long time watching TV with the confidence of
98%>
System: Hello Mr. Johnson, I'm sorry to interrupt you.
Nevertheless, I have noticed that you have watched TV late at night for several days.
Mr. Johnson: I know but I want to finish watching this program.
System: I understand.
<System records the type of the information from the system and the type of the response
from the user>
Sleep assessment
<System notices the recent increasing trend of ‘Napping’ behaviour with the confidence of
87%>
<System also notices frequent ‘Dozing’ behaviour with the confidence of 79%>
System: Hello Mr. Johnson, are you sleeping alright lately?
I noticed that you are dozing off often and taking long naps for several days in the past.
Mr. Johnson: I haven't slept well at night lately.
<System updates the user's wellbeing status related to ‘sleep’ accordingly>
System: I'm sorry to hear that.
<System finds a strong correlation between watching TV late and the lack of sleep of the
user>
System: I wonder if your sleep has been irregular lately because of watching TV late at
night.
Mr. Johnson: I guess that's possible.
<System records the type of the conjecture by the system and the type of the response
from the user>
System: Should I remind you next time if you stay up late?
Mr. Johnson: Sure, that will be helpful.
<System updates the reminder and increases its coercion level>
<System starts to monitor the user's ‘Napping’ and ‘Dozing off’> behaviours closely to find
out if this intervention has any impact on the user>
System: No problem. I will do that.
Is there anything else I can do for you to help you to sleep better?
Mr. Johnson: Not really. Thanks for your concern anyway.
System: I'm always here for you when you need someone to talk to.
Mr. Johnson: Thanks.
System: You are very welcome.
<System records the type of the suggestion by the system and the type of the response
from the user>
<System increases its verbosity level for the user as the user doesn't seem to mind a long
conversation with the system.>
Intervention as requested
<System notices the user is watching TV late at night with the confidence of 91%>
<System has been told by the user to remind the user about watching TV late>
System: Hello Mr. Johnson, I'm sorry to interrupt you again.
Nevertheless, you have asked to remind you about your TV watching. It is late at night.
Mr. Johnson: Oh, yes. I better go to sleep now.
Thank you.
System: You are welcome. Have a good sleep.
<System records the type of the suggestion by the system and the type of the response
from the user>
```

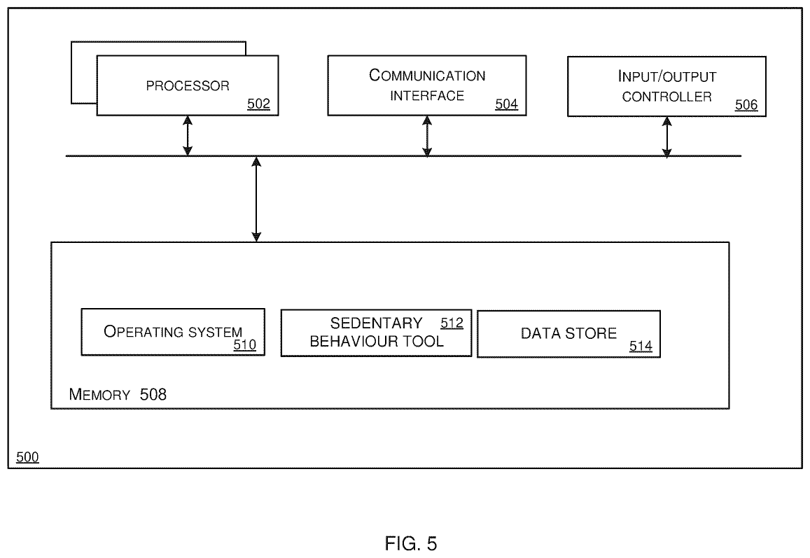

FIG. 5 is a schematic diagram of a computing device implementing a sedentary behaviour tool. FIG. 5 illustrates various components of an example computing device 500 in which embodiments of a sedentary behaviour tool are implemented in some examples. The computing device is of any suitable form such as a smart phone, a desktop computer, a computing device integrated with an image capture device, a tablet computer, a laptop computer.

The computing device 500 comprises one or more processors 502 which are microprocessors, controllers or any other suitable type of processors for processing computer executable instructions to control the operation of the device in order to perform the methods of FIGS. 3 to 4. In some examples, for example where a system on a chip architecture is used, the processors 502 include one or more fixed function blocks (also referred to as accelerators) which implement a part of the method of FIGS. 3 to 4 in hardware (rather than software or firmware). That is, the methods described herein are implemented in any one or more of software, firmware, hardware. The computing device has a data store 514 holding group profile data, behaviour classes, behaviour data, captured sensor data, parameter values or other data. The computing device has a sedentary behaviour tool 512. Platform software comprising an operating system 510 or any other suitable platform software is provided at the computing-based device to enable application software to be executed on the device. Although the computer storage media (memory 508) is shown within the computing-based device 500 it will be appreciated that the storage is, in some examples, distributed or located remotely and accessed via a network or other communication link (e.g. using communication interface 504).

The computing-based device 500 also comprises an input/output controller 506 arranged to output display information to a display device which may be separate from or integral to the computing-based device 500. The display information may provide a graphical user interface and/or audio output. The input/output controller 506 is also arranged to receive and process input from one or more devices, such as a user input device (e.g. a mouse, keyboard, camera, microphone or other sensor).

Any reference to ‘an’ item refers to one or more of those items. The term ‘comprising’ is used herein to mean including the method blocks or elements identified, but that such blocks or elements do not comprise an exclusive list and an apparatus may contain additional blocks or elements and a method may contain additional operations or elements. Furthermore, the blocks, elements and operations are themselves not impliedly closed.

The steps of the methods described herein may be carried out in any suitable order, or simultaneously where appropriate. The arrows between boxes in the figures show one example sequence of method steps but are not intended to exclude other sequences or the performance of multiple steps in parallel. Additionally, individual blocks may be deleted from any of the methods without departing from the spirit and scope of the subject matter described herein. Aspects of any of the examples described above may be combined with aspects of any of the other examples described to form further examples without losing the effect sought. Where elements of the figures are shown connected by arrows, it will be appreciated that these arrows show just one example flow of communications (including data and control messages) between elements. The flow between elements may be in either direction or in both directions.

Where the description has explicitly disclosed in isolation some individual features, any apparent combination of two or more such features is considered also to be disclosed, to the extent that such features or combinations are apparent and capable of being carried out based on the present specification as a whole in the light of the common general knowledge of a person skilled in the art, irrespective of whether such features or combinations of features solve any problems disclosed herein. In view of the foregoing description it will be evident to a person skilled in the art that various modifications may be made within the scope of the invention.

1. A computer-implemented method for monitoring sedentary behaviours of a person for wellbeing, comprising:
receiving, from at least one capture device, captured sensor data depicting at least one sedentary person, and values of configuration parameters of the capture device;
processing the captured sensor data to compute skeletal data of the person;
inputting the skeletal data to a classifier to obtain classification data, for a plurality of classes related to behaviour of the person;
storing the configuration parameters in association with the classification data;
detecting patterns in the stored classification data;
adjusting the values of the configuration parameters using the detected patterns.

2. The method of claim 1 comprising using automated dialog functionality to carry out a dialog with the person taking into account the detected patterns and the classification data.

3. The method of claim 2 comprising adjusting the automated dialog functionality using the detected patterns.

4. The method of claim 1, wherein the configuration parameters are any of: focus, direction, exposure, zoom level.

5. The method of claim 1, wherein adjusting the values of the configuration parameters using the detected patterns comprises ensuring the configuration parameters are suitable for detecting the patterns efficiently.

6. The method of claim 1 comprising selecting a group profile by comparing the skeletal data with a plurality of group profiles.

7. The method of claim 6 comprising adjusting the configuration parameters according to the selected group profile.

8. The method of claim 6 comprising sending data about the selected group profile to an automated dialog functionality in order to facilitate wellbeing dialog with the person.

9. The method of claim 1 wherein processing the captured sensor data to compute the skeletal data comprises computing a plurality of skeleton points; and wherein the method comprises adjusting the number of skeleton points using the detected patterns.

10. The method of claim 1 comprising inputting context data to the classifier together with the skeletal data in order to obtain the classification data.

11. The method of claim 1 comprising using the skeletal data to recognize the person.

12. The method of claim 1 comprising using the skeletal data to detect more than one person and to store data about social interaction events when more than one person is present.

13. A computer program comprising instructions which when executed on a computing device implement the method of claim 1.

14. An apparatus for monitoring sedentary behaviours of a person for wellbeing comprising: means for receiving, from at least one capture device, captured sensor data depicting at least one sedentary person, and values of configuration parameters of the capture device;
a processor configured to process the captured sensor data to compute skeletal data of the person;
a classifier for classifying the skeletal data to obtain classification data, for a plurality of classes related to behaviour of the person;
a memory storing the configuration parameters in association with the classification data; means for detecting patterns in the stored classification data;
means for adjusting the values of the configuration parameters using the detected patterns.

15. The apparatus of claim 14 which is a resource-constrained edge computing device.
